(19) United States
(12) Patent Application Publication
(10) Pub. No.: US 2026/0108461 A1
(43) Pub. Date: Apr. 23, 2026

(54) OIL IN WATER MICROEMULSION FORMULATION

(71) Applicant: MED BioDevelopment Inc., Indianapolis, IN (US)

(72) Inventors: Robert Luft, Indianapolis, IN (US); Kyle Eric Michelson, Indianapolis, IN (US)

(73) Assignee: MED BioDevelopment Inc., Indianapolis, IN (US)

(21) Appl. No.: 18/922,500

(22) Filed: Oct. 22, 2024

(51) Int. Cl. A61K 9/107; A61K 8/06; A61K 31/355; A61K 31/375; A61K 45/06; A61K 47/10; A61K 47/14; A61K 47/22; A61K 47/36; B82Y 5/00

(52) U.S. Cl. CPC A61K 9/1075; A61K 8/062; A61K 8/068; A61K 31/355; A61K 31/375; A61K 45/06; A61K 47/10; A61K 47/14; A61K 47/22; A61K 47/36; A61K 2800/21; A61K 2800/805; B82Y 5/00

(57) ABSTRACT

The present invention relates to a microemulsion formulation comprising oils, non-ionic surfactant, water, and the active ingredient, wherein this microemulsion is produced by mixing an aqueous phase and a lipid phase in which each phase contains at least one surfactant in a defined ratio, at least one amphiphilic co-surfactant, and at least one co-solvent whereby the resulting microemulsion has very small dispersed droplets, is preserved against microbial contamination, and, is thermodynamically stable for long periods of time and at elevated temperature.

### FIELD OF INVENTION

The present invention relates to a microemulsion formulation comprising oils, non-ionic surfactant, water, and active ingredients, wherein this microemulsion is produced by mixing an aqueous phase and a lipid phase in which each phase contains at least one surfactant in a defined ratio whereby the resulting microemulsion has very small, dispersed droplets, and is thermodynamically stable.

### BACKGROUND

Microemulsions are clear, thermodynamically stable, low-viscosity dispersions consisting of two immiscible liquids (typically oil and water) stabilized by a surfactant, often in conjunction with a co-surfactant (e.g. a short-chain alcohol). These formulations are characterized by their ability to form spontaneously without significant energy input, appearing transparent or opalescent due to the small size of their dispersed phase particles, generally ranging from 10 to 200 nm. They exhibit a macroscopically homogeneous and optically isotropic nature, though anisotropic structures can be detected using advanced techniques like X-ray irradiation. Unlike nanoemulsions, which are only kinetically stable, microemulsions reach a thermodynamic equilibrium that prevents phase separation without the need for extensive mechanical energy, allowing them to form spontaneously upon gentle mixing of the components.

In the pharmaceutical and cosmetic industries, microemulsion preparations provide enhancement of skin penetration of various drugs and active ingredients by disrupting the lipid bilayer structure of the stratum corneum, thereby facilitating drug or nutrient transport. This is achieved through the interaction between the lipophilic domains of the microemulsions and the lipids of the stratum corneum. Moreover, the hydration of corneocytes leads to further disruption of lipid bilayers, enhancing the efficacy of transdermal delivery systems.

In cosmetic applications, they are valued for their clear or slightly translucent appearance, providing a fresh sensation due to the external aqueous phase, while also potentially offering advantages in terms of oil content for nourishing the skin. Microemulsions are structurally complex, often described as bi-continuous domains of oil and water stabilized by a saturated surfactant monolayer, with fluctuating domain sizes observed between 3 nm and 100 nm. These unique characteristics make microemulsions suitable for a wide range of applications in cosmetic and dermatological products, like lotions, creams, and serums, where their stability and aesthetic properties are particularly beneficial.

Despite their many advantages, including case of preparation and enhanced stability compared to traditional emulsions, microemulsions can present some challenges. They typically require a high concentration of surfactants, which can lead to skin intolerance, irritation, and a sticky residue upon application. Moreover, their formulation space is generally narrow, and their stability can be temperature dependent.

The choice of the surfactant is another important factor that influences the stability of the microemulsion. Anionic and cationic surfactants cause skin irritation and sometimes sensitization, especially in people with sensitive skin. Hence the preferred choice of surfactant is non-ionic. However, commonly used preservatives like most parabens and phenoxyethanol are inactivated by non-ionic surfactants like polyethylene glycol derivatives of hydrogenated castor oil (Kolliphor RH40) and polyoxyethylene sorbitan esters (Polysorbate 80). Benzoic acid, sorbic acid, benzalkonium chloride, and methylparaben (the least affected paraben) have reduced antimicrobial activity in the presence of non-ionic surfactants.

The stability of the microemulsion is of utmost importance and there are several challenges for the formulator to formulate a stable microemulsion. One such challenge is the stability over a wide range of oil:water:surfactant ratios. Another challenge is the long-term thermodynamic stability of the microemulsion. In case of cosmetic and/or pharmaceutical microemulsions, microbial stability is of utmost importance and but challenging to achieve. A further challenge in case of cosmetic and/or pharmaceutical microemulsions is selecting the right surfactant which would render the microemulsion stable over a wide range of oil:water ratio and is yet compatible with the other ingredients including the active ingredient.

Thus, the object of the present invention, was to formulate a microemulsion composition which had the widest range of oil:water:surfactant ratio, is thermodynamically stable long-term at elevated temperatures (e.g. 3 months at 40 degrees C.), can remain stable in the presence of multiple active ingredients, and is resistant to microbial spoilage without the need for traditional preservatives such as parabens or phenoxyethanol.

### SUMMARY OF THE INVENTION

Thus, we have surprisingly found that the present invention, a cosmetic or pharmaceutical oil in water (o/w) microemulsion composition comprising,
a. a lipid phase comprising an oily component and one or more surfactant(s) and one or more co-surfactant(s),
b. an aqueous phase comprising one or more non-ionic surfactant(s), and one or more co-surfactants; and
c. at least one co-solvent,
   wherein the microemulsion has an average particle size of less than 100 nm, is thermodynamically stable at elevated temperature, capable of containing multiple active ingredients with cosmetic and/or pharmaceutical relevance, resistant to microbial spoilage and has the widest range of oil:water:surfactant ratio.

In one aspect, the present invention relates to a cosmetic or pharmaceutical oil in water (o/w) microemulsion composition comprising,
a. a lipid phase comprising an oily component and one or more surfactant(s) and one or more co-surfactant(s),
b. an aqueous phase comprising one or more non-ionic surfactant(s), and one or more co-surfactants; and
c. at least one co-solvent,
   wherein the microemulsion has an average particle size of less than 100 nm.

In a second aspect, the present invention relates to a cosmetic or pharmaceutical oil in water (o/w) microemulsion composition comprising,
a. a lipid phase comprising i) 5-20% w/w of an oily component, ii) 5-10% w/w of one or more lipophilic non-ionic surfactants;
b. an aqueous phase comprising i) 50-90% w/w of water, ii) 5-20% w/w of a hydrophilic non-ionic surfactant;
c. 1-10% w/w of co-surfactants; and
d. 1-5% w/w of a co-solvent;
   wherein the composition has an average particle size of less than 100 nm.

In a third aspect, the present invention relates to a cosmetic or pharmaceutical oil in water (o/w) microemulsion composition comprising,
a. a lipid phase comprising an oily component and one or more surfactant(s) and one or more co-surfactant(s),
b. an aqueous phase comprising one or more non-ionic surfactant(s), and one or more co-surfactants; and
c. at least one co-solvent,
   wherein the microemulsion has an average particle size of less than 100 nm, wherein the ratio of oil:water:surfactant is in the range of 1-3:10-20:1-5.

In a fourth aspect, the present invention relates to a method of preparing the cosmetic or pharmaceutical oil in water (o/w) microemulsion composition, comprising the steps:
preparing the lipid phase,
preparing the aqueous phase,
combining the lipid phase and the aqueous phase, adding to the mixture a co-solvent, active ingredients, preservative, and other excipients, and
stirring the mixture continuously to form an oil in water (o/w) microemulsion having pH 4.0-7.0 and an average particle size less than 100 nm.

In a fifth aspect, the present invention relates to the use of the oil in water (o/w) microemulsion according to the present invention as an ingredient for preparing, cosmetics, personal care products, in particular skin cleaning products, shampoos, rinse-off conditioners, deodorants, antiperspirants, body lotions, and pharmaceutical compositions.

In a sixth aspect, the present invention relates to an oil in water (o/w) microemulsion composition comprising skin actives selected from Vitamin A and its derivatives, Vitamin B5 and its derivatives, Vitamin C and its derivatives, Vitamin E and its derivatives, Provitamins, Niacinamide, Hyaluronic acid, Ceramides, Benzoyl peroxide, Kojic Acid, Azelaic acid, Ferulic acid, Alpha-Hydroxy Acids (AHAs), Beta-Hydroxy Acids (BHAs), Polyhydroxy Acid (PHAs), Acetyl zingerone, Hydroquinone, other skin active agent, or a combination thereof.

### DETAILED DESCRIPTION OF THE INVENTION

As used herein, a microemulsion is a two-phase system composed of a lipid phase and an aqueous phase, finely interspersed. Microemulsions are liquid mixtures, transparent, isotropic and stable, of a lipid phase and an aqueous phase, held together by a surfactant, generally in conjunction with a co-surfactant. Microemulsions are clear, thermodynamically stable, isotropic liquid mixtures of oil, water and surfactant, and optionally a co-surfactant. Microemulsions form spontaneously upon mixing of the aqueous phase and lipid phase. A microemulsion is, therefore, a thermodynamically stable system, with particles dispersed in the continuous phase. The particle size of the dispersed phase in a microemulsion is less than 100 nm, generally in the range between 5 nm and 50 nm, inclusive.

As used herein, “particle size” and “average particle size” refer synonymously to the average diameter of particles in a provided liquid, for example, the droplet diameter or micelle diameter in an emulsion.

As used herein, “oil phase” or “lipid phase” is used to refer to the portion (or phase) of a composition such as those provided herein that contains one or more lipophilic ingredients and/or amphiphilic ingredients, such as an oil, and is, in general, the lipid-soluble phase. In an oil in water (o/w) microemulsion, the lipid phase typically is the dispersed phase while water is the dispersion phase.

As used herein, “water phase” or “aqueous phase” refers to the portion (phase) of a composition, such as those provided herein, that contains one or more hydrophilic ingredients and/or amphiphilic ingredients (water phase ingredients) and is, in general, the water-soluble phase. Typically, in the microemulsion compositions provided herein, the water phase is the continuous phase. “Water phase” also is used to refer to the liquid containing the water phase ingredients that is generated while preparing microemulsions.

As used herein, water phase ingredient(s) refers to the components of the compositions that are included in the water phase in the provided methods for making the compositions. Typical water phase ingredients can include, but are not limited to, polar solvents, typically polar protic solvents, such as water and alcohols, typically alcohols having more than one hydroxy group such as dihydroxy and trihydroxy alcohols, such as glycerol and propylene glycol; at least one surfactant; preservatives; and emulsion stabilizers. Other hydrophilic and/or amphiphilic ingredients can be included in the water phase.

As used herein, “surfactant” refers to synthetic and naturally occurring amphiphilic molecules that have hydrophobic portion(s) and hydrophilic portion(s). Surfactants contain a hydrophilic domain and hydrophobic domain, i.e. amphiphilic molecules. Given their nature, surfactants facilitate the formation of oil in water (o/w) emulsions where the micelles, in order to exist, need to interact with both the water and the oil phases. Due to their amphiphilic (amphipathic) nature, surfactants and co-surfactants can reduce the surface tension between two immiscible liquids, for example, the oil and water phases in an emulsion, such as a microemulsion, stabilizing the emulsion.

Surfactants can be characterized based on their relative hydrophobicity and/or hydrophilicity. For example, relatively lipophilic surfactants are more soluble in fats, oils and waxes, typically having Hydrophobic-Lipophilic Balance (HLB) values less than 10 or about 10, while relatively hydrophilic surfactants are more soluble in aqueous compositions, for example, water, and typically have HLB values greater than 10 or about 10. Relatively amphiphilic surfactants are soluble in oil- and water-based liquids and typically have HLB values close to 10 or about 10. Surfactants for use in the compositions herein are biocompatible and have an HLB value between 8 or about 8 and 16 or about 16, generally 10-16, and most preferably between 14-16.

As used herein, a co-surfactant is a surfactant that acts in addition to another surfactant to further reduce the surface tension of a liquid. Recitation that microemulsions contain surfactants refers to the surfactants and the co-surfactants that are included. Co-surfactants are hydrophilic in nature and reduce the surface tension of water. They generally are used as wetting agents, for example, to increase the spreading abilities of water-based fluid by reducing the surface tension of water. Cosurfactants also are used, and often needed, to increase the solubility of the primary surfactant.

As used herein, thermodynamic stability of the microemulsions refers to the stability of the dispersion such that the phases do not separate over time. The microemulsions provided herein exhibit an unusually high thermodynamic stability as shown by their stability at elevated temperatures of 40° C. for a period exceeding 3 months.

As used herein, room temperature and ambient temperature are used to describe a temperature that is common in one or more enclosed spaces in which human beings typically are or reside. Room temperature can vary, but generally refers to temperatures between 19° C. or about 19° C. and 25° C. or about 25° C. When a composition is stored at room temperature, it should be understood it is generally kept at a temperature within this range or about within this range.

As used herein, the singular forms “a,” “an” and “the” include plural referents unless the context dictates otherwise.

As used herein, ranges and amounts can be expressed as “about” a particular value or range. About also includes the exact amount. Hence “about 5 grams” means “about 5 grams” and also “5 grams”. It also is understood that ranges expressed herein include whole numbers within the ranges and fractions thereof. For example, a range of between 5 grams and 20 grams includes whole number values such as 5, 6, 7, 8, 9, 10, 11, 12, 13, 14, 15, 16, 17, 18, 19 and 20 grams, and fractions within the range, for example, but not limited to, 5.25, 6.72, 8.5, and 11.95 grams.

As used herein, “optional” or “optionally” means that the subsequently described event or circumstance does or does not occur, and that the description includes instances where said event or circumstance occurs and instances where it does not. For example, an optionally variant portion means that the portion is variant or non-variant.

Provided herein are thermodynamically stable microemulsions that can be used to solubilize active compounds. The microemulsions provided herein, advantageously, can be used for cosmetic and/or pharmaceutical applications. The microemulsions are prepared from: a lipid phase containing an oily component and one or more surfactants and co-surfactants; and an aqueous phase, containing one or more surfactants and co-surfactants. In some embodiments the aqueous phase contains one surfactant and one co-surfactant. Additional ingredients used in emulsions and compositions for pharmaceutical use also can be included.

As described herein the microemulsions provided herein have small particles of relatively uniform size. As a result, the emulsions are very stable and can be used to formulate pharmaceutically active compounds to produce very stable compositions in which the compounds have high bioavailability.

The oil in water (o/w) microemulsions are formed by including an oil in the lipid phase, and at least one surfactant and one co-surfactant in each of the aqueous and lipid phases in precise ratios. When these ratios are used, the microemulsions that form upon mixing the two phases, such as by titrating the lipid phase with the aqueous phase (adding the aqueous phase into the lipid phase), have a very narrow distribution range of very small particles (<100 nm). As a result, the microemulsions are unusually stable, and can be used to formulate active compounds that have limited aqueous solubility.

In an embodiment, the present invention, relates to a cosmetic or pharmaceutical oil in water (o/w) microemulsion composition comprising,
a. a lipid phase comprising an oily component and one or more surfactant(s) and one or more co-surfactant(s),
b. an aqueous phase comprising one or more non-ionic surfactant(s), and one or more co-surfactants; and
c. at least one co-solvent,
   wherein the microemulsion has an average particle size of less than 100 nm

In another embodiment, the present invention is a cosmetic or pharmaceutical oil in water (o/w) microemulsion comprising,
a. a lipid phase comprising i) 5-20% w/w of an oily component, ii) 5-10% w/w of one or more lipophilic non-ionic surfactants;
b. an aqueous phase comprising i) 50-90% w/w of water, ii) 1-10% w/w of a hydrophilic non-ionic surfactant;
c. 1-10% w/w of co-surfactants; and
d. 1-5% w/w of a co-solvent;
   wherein the microemulsion has an average particle size of less than 100 nm.

Further details of the components of the microemulsions, including the oils, surfactants, co-surfactants, co-solvents, active compounds, aqueous phase, additives etc. are described herein.

### Oils

The composition according to the present invention comprises at least one oil. Here, “oil” means a fatty compound or substance which is in the form of a liquid at room temperature (25° C.) As the (a) oil(s), those generally used in cosmetics can be used alone or in combination thereof. The oil(s) may be volatile or non-volatile, preferably non-volatile.

The (a) oil may be a non-polar oil such as a hydrocarbon oil, a silicone oil, or the like; a polar oil such as a plant or animal oil and an ester oil or an ether oil; or a mixture thereof.

It is preferable that the (a) oil be selected from the group consisting of oils of plant or animal origin, synthetic oils, silicone oils and hydrocarbon oils.

As examples of plant oils, mention may be made of, for example, coconut oil, linseed oil, camellia oil, palm kernel oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof.

As examples of animal oils, mention may be made of, for example, squalene and squalane.

As examples of synthetic oils, mention may be made of alkane oils such as isododecane and isohexadecane, ester oils, ether oils and artificial triglycerides.

The ester oils are preferably liquid esters of saturated or unsaturated, linear or branched C1-C26 aliphatic monoacids or polyacids and of saturated or unsaturated, linear or branched C1-C26 aliphatic monoalcohols or polyalcohols, the total number of carbon atoms of the esters being less than or equal to 10.

Preferably, for the esters of monoalcohols, at least one from among the alcohol and the acid from which the esters of the invention are derived is branched.

Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate and isostearyl neopentanoate.

Esters of C4-C22 dicarboxylic or tricarboxylic acids and of C1-C22 alcohols and esters of monocarboxylic, dicarboxylic or tricarboxylic acids and of non-sugar C4-C26 dihydroxy, trihydroxy, tetrahydroxy or pentahydroxy alcohols may also be used.

Mention may especially be made of—diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, ethylhexyl palmitate, isohexyl laurate, hexyl laurate, isocetyl stearate, isopropyl isostearate, isopropyl myristate, isodecyl oleate, glyceryl tri (2-ethylhexanoate), pentaerythrithyl tetra(2-ethylhexanoate), 2-ethylhexyl succinate, diethyl sebacate, and mixtures thereof.

As examples of ether oils, mention may be made of, for example, dicaprylylether and diisocetylether.

As examples of artificial triglycerides, mention may be made of, for example, glyceryl trimyristate, glyceryl tripalmitate, glyceryl trilinolenate, glyceryl trilaurate, glyceryl tricaprate, glyceryl tricaprylate, glyceryl tri (caprate/caprylate) and glyceryl tri (caprate/caprylate/linolenate).

Hydrocarbon oils may be chosen from:
linear or branched, optionally cyclic, C6-C16 lower alkanes. Examples that may be mentioned include hexane, undecane, dodecane, tridecane, and isoparaffins, for instance isohexadecane, isododecane and isodecane; and linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as mineral oil (e.g., liquid paraffin), paraffin, vaseline or petrolatum, naphthalenes, and the like; hydrogenated polyisobutene, isocicosan, and decene/butene copolymer; and mixtures thereof.

The (a) oil may be a fatty alcohol. The term “fatty alcohol” here means any saturated or unsaturated, linear or branched C8-C30 alcohol, which is optionally substituted, in particular, with one or more hydroxyl groups (in particular 1 to 4). If they are unsaturated, these compounds may comprise one to three conjugated or non-conjugated carbon-carbon double bonds.

Among the C8-C30 fatty alcohols, C12-C30 fatty alcohols, for example, may be used. Mention may be made, among these, of lauryl alcohol, cetyl alcohol, stearyl alcohol, isostearyl alcohol, oleyl alcohol, behenyl alcohol, linoleyl alcohol, undecylenyl alcohol, palmitoleyl alcohol, linolenyl alcohol, myristyl alcohol, arachidonyl alcohol, erucyl alcohol, octyldodecanol, and mixtures thereof.

Preferably, the (a) oil has a low molecular weight such as below 600 g/mol, chosen among ester oils with a short hydrocarbon chain or chains (C1-C12) (e.g., isopropyl myristate, isopropyl palmitate, isononyl isononanoate, and ethyl hexyl palmitate), hydrocarbon oils (e.g., isododecane, isohexadecane, and squalane), branched and/or unsaturated fatty alcohol (C12-C30) type oils such as octyldodecanol and oleyl alcohol, and ether oils such as dicaprylylether.

The amount in the composition according to the present invention of the (a) oil is not limited, and may range from 5 to 30% by weight, preferably from 5 to 20% by weight, and more preferably from 5 to 15% by weight, relative to the total weight of the composition.

In an embodiment, the oily component is selected from fatty acid or polyol esters of animal or vegetable oil, C6-C12 medium chain triglycerides (MCT oil), caprylic/capric triglyceride, or combination thereof, most preferably made from C6-C10 MCT oil.

### Surfactants

In an embodiment, for preparing the microemulsions each phase must include at least one surfactant, and the system will generally include at least one hydrophilic or amphiphilic co-surfactant. The surfactant(s) in the lipid and aqueous phases can be the same or different.

In an embodiment, the present invention includes a non-ionic surfactant.

Surfactants can be selected from among, for example, poloxamers, PEGylated fatty acids, polyoxyethylene sorbitan fatty acid derivatives, polyoxyethylenes, hydrogenated castor oil ethoxylates, glycerol esters of fatty acids, polyoxyl castor oil surfactants, amine oxides, and alcohol ethoxylates (non-ionic). Exemplary of these are polyethylene glycol sorbitan monolaurate (Polysorbate 20; Tween 20), polyethylene glycol sorbitan monooleate (Polysorbate 80; Tween 80), and polyethylene glycol sorbitan monopalmitate (Polysorbate 40; Montanox 40).

In other embodiments the surfactants are selected from among polyoxyl 35 castor oil (Cremophor EL, Kolliphor EL), polyoxyl 40 hydrogenated castor oil (Cremophor RH40; Kolliphor RH 40), PEG 40 castor oil (Etocas 40), PEG-60 hydrogenated castor oil (Croduret 60), and polyethylene glycol 15-hydroxystearate (Kolliphor HS 15).

The surfactants can be non-ionic. For example, non-ionic surfactants can be selected from among Pluronic®, Cremophor®, Kolliphor®, Polysorbates (Tween™), lauryl dimethyl ammine oxide, polyethoxylated alcohol, polyoxyl lauryl ether, Brij®, polyoxyethylated castor oil, poloxamers, polyethylene glycol, glycerol esters of fatty acids. In some embodiments the surfactants are selected from among castor oil or hydrogenated castor oil ethoxylates.

In an embodiment, the non-ionic surfactant of the lipid phase is selected from glyceryl caprylate/caprate, mono- and di-glyceride of medium chain fatty acids, polyglycerol fatty acid ester, caprylic/capric acid polyethylene glycol glyceride, or a combination thereof.

In an embodiment, non-ionic surfactant of the lipid phase is used in the range of 5% w/w to 20% w/w based on the total weight of the composition.

In an embodiment, the non-ionic surfactant of the aqueous phase is selected from hydrogenated animal or vegetable oil and its derivatives, polyoxyl 40 hydrogenated castor oil, sorbitan fatty acid ester, polyoxyethylene sorbitan fatty acid ester, fatty alcohol polyoxyethylene ether, poloxamers, or a combination thereof.

In an embodiment, non-ionic surfactant of the aqueous phase is used in the range of 1% w/w to 20% w/w based on the total weight of the composition.

### Co-Surfactant

A co-surfactant is a surfactant that acts in addition to another surfactant to further reduce the surface tension of a liquid. Co-surfactants in oil in water (o/w) microemulsions are generally amphiphilic or slightly hydrophilic and they reduce the surface tension of water. They generally are used as wetting agents, for example, to increase the spreading abilities of water-based fluid by reducing the surface tension of water. Cosurfactants also are used, and often needed, to increase the solubility of the primary surfactant.

In an embodiment, the co-surfactants are selected from the group comprising of C8-C20 monoglyceryl ester, C8-C20 diglyceryl ester, C4-C8 1,2-alkanediols selected from 1,2-butanediol (C4), 1,2-pentanediol (C5), 1,2-hexanediol (C6), 1,2-octanediol (C8), or a combination thereof.

In an embodiment, the co-surfactants are preferably C4-C8 1,2-alkanediols selected from 1,2-butanediol (C4), 1,2-pentanediol (C5), 1,2-hexanediol (C6), 1,2-octanediol (C8), or a combination thereof.

In an embodiment, the co-surfactant is used in the range of 1% w/w to 10% w/w based on the total weight of the composition.

### Co-Solvent

In an embodiment, the co-solvent is selected from low molecular weight mono- or poly-oxy-alkane diol comprising alkyl derivatives, selected from ethoxydiglycol, diethylene glycol monoethyl ether, diethylene glycol monomethyl ether, diethylene glycol mono-n-propyl ether, diethylene glycol mono-iso-propyl ether, diethylene glycol mono-n-butyl or mono-iso-butyl ether, diethylene glycol mono-n-hexyl ether, or a combination thereof.

In an embodiment, the co-solvent is used in the range of 1% w/w to 5% w/w based on the total weight of the composition.

### Aqueous Phase

In an embodiment, the aqueous phase comprises water and may be, for example, a mixture of water and hydrophilic compound(s), especially such as polyhydric alcohols, for instance glycerol, propylene glycol, dipropylene glycol and sorbitol, water-soluble lower alcohols such as ethanol, isopropanol or butanol. In addition, it can, of course, contain water-soluble or water-dispersible adjuvants, and in particular water-soluble adjuvants usually used in the fields under consideration.

In an embodiment, the aqueous phase is in the range of 50% to 90% by weight relative to the total weight of the composition, preferably from 55% to 75% by weight relative to the total weight of the composition.

### Active Agent

In an embodiment, the oil in water (o/w) microemulsion includes at least one active agent which may be selected from:
vitamins, for instance vitamins A and C mentioned above, and also vitamin E (tocopherol) and its derivatives; vitamin B3 (or vitamin PP or niacinamide) and its derivatives; vitamin B5 (or panthenol or panthenyl alcohol or 2,4-dihydroxy-N-(3-hydroxypropyl)-3,3-dimethylbutanamide), in its various forms: D-panthenol, DL-panthenol), and derivatives and analogues thereof, such as calcium panthotenate, panthetine, pantotheine, ethyl panthenyl ether, pangamic acid, pyridoxine, pantoyl lactose and natural compounds containing it such as royal jelly; vitamin D and its analogues; mixtures of unsaturated acids containing at least one double bond and especially mixtures of linoleic acid, of linolenic acid and of arachidonic acid, or compounds containing them;
anti-aging active agents and keratolytic agents such as α-hydroxy acids and especially acids derived from fruit, for instance glycolic acid, lactic acid, malic acid, citric acid, tartaric acid and mandelic acid, derivatives thereof, and mixtures thereof; β-hydroxy acids, for instance salicylic acid and its derivatives such as 5-n-octanoylsalicylic acid or 5-n-dodecanoylsalicylic acid; α-keto acids, for instance ascorbic acid or vitamin C and its derivatives such as its salts, for instance sodium ascorbate and magnesium or sodium ascorbyl phosphate; its esters, for instance ascorbyl acetate, ascorbyl palmitate and ascorbyl propionate, or its sugars, for instance glycosylated ascorbic acid, and mixtures thereof; β-keto acids; retinoids, for instance retinol (vitamin A) and its esters, retinal, retinoic acid and its derivatives, adapalene, carotenoids, and mixtures thereof;
moisturizers, for example sodium lactate; polyols, and in particular glycerol, sorbitol and polyethylene glycols; mannitol; amino acids; hyaluronic acid; lanolin; urea and mixtures containing urea, such as NMF (Natural Moisturizing Factor); petroleum jelly; N-lauroyl pyrrolidone carboxylic acid and its salts; essential fatty acids; essential oils; and mixtures thereof;
antibacterial agents and anti-seborrhoeic agents such as salicylic acid, 2,4,4′-trichloro-2′-hydroxydiphenyl ether (or triclosan), 3,4,4′-trichlorobanilide (or triclocarban), azelaic acid, benzoyl peroxide and zinc salts, for instance zinc lactate, zinc gluconate, zinc pidolate, zinc carboxylate, zinc salicylate and/or zinc cysteate.

In an embodiment, the at least one active agent includes vitamins and derivatives thereof, such as vitamin E, vitamin E acetate, vitamin C and its esters, B vitamins, vitamin A alcohol and vitamin A retinol, vitamin A acid and vitamin A retinoic acid and its derivatives, provitamins such as panthenol, vitamin A palmitate, niacinamide, antioxidants, essential oils, wetting agents, preserving agents, sequestering agents, softeners, dyes, viscosity modifiers, pigments, moisturizers, proteins, ceramides, pseudoceramides, fatty acids comprising linear and branched C16-C40 chains, such as 18-methyl eicosanoic acid, and hydroxy acids, electrolytes, polymers, such as cationic polymers.

In an embodiment, the at least one active agent is selected from Vitamin A and its derivatives, Vitamin B5 and its derivatives, Vitamin C and its derivatives, Vitamin E and its derivatives, Provitamins, Niacinamide, Hyaluronic acid, Ceramides, Benzoyl peroxide, Kojic Acid, Azelaic acid, Ferulic acid, Alpha-Hydroxy Acids (AHAs), Beta-Hydroxy Acids (BHAs), Polyhydroxy Acid (PHAs), Acetyl zingerone, Hydroquinone, other skin active agent, or a combination thereof.

In an embodiment, the at least one active agent is present in the range of 1% w/w to 5% w/w based on the total weight of the composition.

### Preservative

In an embodiment, the composition comprises a preservative. In an embodiment, the preservative is selected from medium chain mono- and diglycerides of fatty acids, C6-C10 alkanediols, polyhydric alcohols, alkylhydroxamic acid or a salt thereof, or a combination thereof. In an embodiment, the composition comprises a preservative that is not a paraben or phenoxyethanol.

In another embodiment, the preservative is present in the range of 0.5 wt. % to 5 wt % based on the total weight of the composition.

### Other Excipients

In an embodiment, the composition may contain other adjuvants in the aqueous phase and/or in the oily phase of the emulsions in accordance with the invention (according to their water-soluble or liposoluble nature). In an embodiment, the excipients mentioned may be selected from ionic or non-ionic thickeners, tonicity agents, antioxidants, stabilizing agents, buffering agents, preserving agents, fillers, sequestering agents, or any other ingredient usually used in the fields under consideration.

### Particle Size

In an embodiment, the particle size of the droplet is measured using a Zetasizer Nano ZS from Malvern Panalytical.

In an embodiment, the particle size of the microemulsion is less than 100 nm. In another embodiment, the particle size of the microemulsion is less than 90 nm. In another embodiment, the particle size of the microemulsion is less than 80 nm. In another embodiment, the particle size of the microemulsion is less than 70 nm.

In another embodiment, the particle size of the microemulsion is less than 60 nm. In another embodiment, the particle size of the microemulsion is less than 50 nm.

pH

In an embodiment, the aqueous phase may also include buffers such as, but not limited to, citric acid and potassium/sodium citrate, disodium EDTA and tetrasodium EDTA, disodium EDTA and disodium phosphate.

In an embodiment, the buffer included in the aqueous phase is a citrate buffer.

In an embodiment, the pH of the microemulsion composition is in the range of 4.0 to 7.0, preferably in the range of 4.0 to 6.0.

In an embodiment, the pH of the microemulsion composition is in the range of 4.0 to 5.0.

### Process for the Preparation of the Microemulsion

In an embodiment, the process for preparing the cosmetic or pharmaceutical oil in water (o/w) microemulsion comprises the steps of:
A. preparing the lipid phase by mixing
   i) the oily component selected from fatty acid or polyol esters of animal or vegetable oil, C6-C12 medium chain triglycerides (MCT oil), caprylic/capric triglyceride, or combination thereof;
   ii) the non-ionic surfactant of the lipidic phase selected from glyceryl caprylate/caprate, mono- and di-glyceride of medium chain fatty acids, polyglycerol fatty acid ester, caprylic/capric acid polyethylene glycol glyceride, or a combination thereof;
   iii) the oil soluble skin actives selected from Vitamin A and its derivatives, Vitamin B5 and its derivatives, Vitamin C and its derivatives, Vitamin E and its derivatives, Provitamins, Ceramides, Benzoyl peroxide, Azelaic acid, Ferulic acid, Beta-Hydroxy Acids (BHAs), Acetyl zingerone, other skin active agent, or a combination thereof;
B. preparing the aqueous phase by mixing
   i) water;
   ii) the non-ionic surfactant of the aqueous phase selected from hydrogenated animal or vegetable oil and its derivatives, polyoxyl 40 hydrogenated castor oil, sorbitan fatty acid ester, polyoxyethylene sorbitan fatty acid ester, fatty alcohol polyoxyethylene ether, poloxamers, or a combination thereof;
   iii) the water-soluble skin actives selected from Vitamin B5 and its derivatives, Vitamin C and its derivatives, Niacinamide, Hyaluronic acid, Ceramides, Kojic Acid, Ferulic acid, Alpha-Hydroxy Acids (AHAs), Polyhydroxy Acid (PHAs), Hydroquinone, other skin active agent, or a combination thereof;
   iv) the stabilizing and buffer agents;
C. combining the lipid phase, the aqueous phase,
D. adding to the mixture
   i) the co-solvent selected from low molecular weight mono- or poly-oxy-alkane diol comprising alkyl derivatives, selected from ethoxydiglycol, diethylene glycol monoethyl ether, diethylene glycol monomethyl ether, diethylene glycol mono-n-propyl ether, diethylene glycol mono-iso-propyl ether, diethylene glycol mono-n-butyl or mono-iso-butyl ether, diethylene glycol mono-n-hexyl ether, or a combination thereof;
   ii) the co-surfactant selected from C4-C8 1,2-alkanediols selected from 1,2-butanediol (C4), 1,2-pentanediol (C5), 1,2-hexanediol (C6), 1,2-octanediol (C8), or a combination thereof;
   iii) the preservatives selected from medium chain mono- and diglycerides of fatty acids, C6-C10 alkanediols, polyhydric alcohols, alkylhydroxamic acid or a salt thereof, or a combination thereof;
   iv) the excipients selected from tonicity agent, anti-oxidant, stabilizing agent, or a combination thereof; and
E. stirring the mixture continuously to form an oil in water (o/w) microemulsion having pH 4.0-7.0 and an average particle size less than 100 nm.

In an embodiment the oil in water (o/w) microemulsion comprises:
a. a lipid phase comprising,
   the oily component selected from fatty acid or polyol esters of animal or vegetable oil, C6-C12 medium chain triglycerides (MCT oil), caprylic/capric triglyceride, or combination thereof;
   the non-ionic surfactant of the lipidic phase selected from glyceryl caprylate/caprate, mono- and di-glyceride of medium chain fatty acids, polyglycerol fatty acid ester, caprylic/capric acid polyethylene glycol glyceride, or a combination thereof;
b. an aqueous phase comprising
   water;
   the non-ionic surfactant of the aqueous phase selected from hydrogenated animal or vegetable oil and its derivatives, polyoxyl 40 hydrogenated castor oil, sorbitan fatty acid ester, polyoxyethylene sorbitan fatty acid ester, fatty alcohol polyoxyethylene ether, poloxamers, or a combination thereof;
   the co-surfactant selected from C4-C8 1,2-alkanediols selected from 1,2-butanediol (C4), 1,2-pentanediol (C5), 1,2-hexanediol (C6), 1,2-octanediol (C8), or a combination thereof;
c. the co-solvent selected from low molecular weight mono- or poly-oxy-alkane diol comprising alkyl derivatives, selected from ethoxydiglycol, diethylene glycol monoethyl ether, diethylene glycol monomethyl ether, diethylene glycol mono-n-propyl ether, diethylene glycol mono-iso-propyl ether, diethylene glycol mono-n-butyl or mono-iso-butyl ether, diethylene glycol mono-n-hexyl ether, or a combination thereof;
d. the skin actives selected from Vitamin A and its derivatives, Vitamin B5 and its derivatives, Vitamin C and its derivatives, Vitamin E and its derivatives, Provitamins, Niacinamide, Hyaluronic acid, Ceramides, Benzoyl peroxide, Kojic Acid, Azelaic acid, Ferulic acid, Alpha-Hydroxy Acids (AHAs), Beta-Hydroxy Acids (BHAs), Polyhydroxy Acid (PHAs), Acetyl zingerone, Hydroquinone, other skin active agent, or a combination thereof;
e. the preservatives selected from medium chain mono- and diglycerides of fatty acids, C6-C10 alkanediols, polyhydric alcohols, alkylhydroxamic acid or a salt thereof, or a combination thereof, and
f. the excipients selected from tonicity agent, anti-oxidant, stabilizing agent, or a combination thereof.

### EXAMPLES

The presently claimed invention is illustrated in detail by non-restrictive working examples which follow.

### Materials

The active ingredients were purchased from commercial sources.

The excipients used were all lab grade.
Kolliphor RH-40 is PEG-40 hydrogenated castor oil
Captex 355 C is medium chain triglycerides (MCT)
Capmul MCM is mono- and di-glycerides of medium chain fatty acids
VitaSynol C is tetrahexyldecyl ascorbate
SymDiol 68 is 1,2-hexanediol/1,2-octanediol
Spectrastat is caprylhydroxamic acid/1,2-octanediol/glycerin
Synoxyl AZ is acetyl zingerone
Transcutol is ethoxydiglycol

### Analysis

Particle size analyser used for the characterization was a Zetasizer Nano ZS from Malvern Panalytical.

### Example 1: Microemulsion Composition I

TABLE 1
Composition of microemulsion I (1000 g batch)

| Sr. No. | Component | w/w % | Mass (g) |
|---|---|---|---|
| 1 | Water | 56.6% | 566 |
| 2 | Citric Acid | 0.16% | 1.6 |
| 3 | Sodium Citrate Tribasic | 0.24% | 2.4 |
| 4 | Kolliphor RH 40 | 13.50% | 135 |
| 5 | Captex 355 C | 9.50% | 95 |
| 6 | Capmul MCM | 9.50% | 95 |
| 7 | VitaSynol C | 2.00% | 20 |
| 8 | Vitamin E Acetate | 1.00% | 10 |
| 9 | D-Panthenyl Triacetate | 1.00% | 10 |
| 10 | SymDiol 68 | 2.00% | 20 |
| 11 | Spectrastat | 1.00% | 10 |
| 12 | Mixed Tocopherols (95%) | 0.50% | 5 |
| 13 | Synoxyl AZ | 0.50% | 5 |
| 14 | Transcutol | 2.50% | 25 |
| | Total | 100% | 1000 |

TABLE 2
Details of Phases in the Composition of microemulsion

| Phase | Description | Composition |
|---|---|---|
| Phase A | Water phase | Kolliphor RH 40, deionized or |
| | | distilled water, citric acid |
| | | anhydrous, Na-citrate tribasic |
| | | dihydrate |
| Phase B | Oil phase | Captex 355 C, Capmul MCM, D- |
| | | Panthenyl Triacetate (B5), |
| | | VitaSynol C, Vitamin E Acetate, |
| | | Mixed Tocopherols |
| Phase C | Co-solvent | Transcutol, Synoxyl AZ |
| Phase D | Preservative | Spectrastat, SymDiol 68 |

### Process of Preparation

### Preparation of Phases

### Phase A

i) Weighed quantities of Citric acid & Sodium citrate tribasic were dissolved in distilled or deionized water at room temperature to make buffer, have pH approximately 4.5. If pH is <4.3 or >4.6 then adjust using NaOH or HCl.
ii) Weighed amount of Kolliphor RH 40 (or similar PEG-40 Castor Oil, HLB=15) was dissolved in a small quantity of the buffered water.
iii) The remaining quantity of the buffer was added to the Kolliphor RH 40 (at room temperature or up to 50° C., until clear) solution and gently mix until transparent.

### Phase B

i) Weighed amounts of Capmul MCM and Captex 355 C were heated to 40° C. in separate containers. The warm Captex 355 C and Capmul MCM were mixed at 40° C. for 10-15 min to form a transparent, homogenous mixture of the two oils.
ii) Once the above mixture was cooled to room temperature, VitaSynol C, D-Panthenyl Triacetate, Vitamin E Acetate, and Mixed Tocopherols were added and mixed until dissolved.

### Phase C

Weighed amount of Synoxyl AZ was added to Transcutol and mixed at 60 rpm for 5-10 minutes until crystals were dissolved, forming a transparent and clear solution devoid of any yellow coloration.

### Phase D

Weighed amounts of Spectrastat and SymDiol 68 were mixed to make a transparent, homogenous mixture (Caprylyl Glycol being a major component of these two preservatives).

### Mixing of Phases

i. Phase C was added to Phase B and mixed at room temperature, thereby dissolving or distributing all the actives in the oil phase.
ii. The mixture containing Phase B and Phase C (formed in the above step i.) was added to Phase A and mixed at room temperature until it formed a clear or translucent, homogenous mixture that remained translucent for at least several minutes, gradually becoming clear. Thus, a microemulsion was formed.
iii. Phase D was added to the mixture formed in step ii above and mixed until a stable, translucent and homogenous mixture was formed that remains translucent for at least several minutes, indicating its stability.

The final product was bottled and stored away from light.

### Example 2: Microemulsion Composition II

TABLE 3
Composition of microemulsion II (1000 g batch)

| | Component | w/w % | Mass (g) |
|---|---|---|---|
| 1 | Water | 58.6% | 586 |
| 2 | Citric Acid | 0.16% | 1.6 |
| 3 | Sodium Citrate Tribasic | 0.24% | 2.4 |
| 4 | Polysorbate 80 | 12.00% | 120 |
| 5 | Captex 355 C | 9.50% | 95 |
| 6 | Capmul MCM | 9.50% | 95 |
| 7 | VitaSynol C | 2.00% | 20 |
| 8 | Vitamin E Acetate | 1.00% | 10 |
| 9 | D-Panthenyl Triacetate | 1.00% | 10 |
| 10 | SymDiol 68 | 2.00% | 20 |
| 11 | Spectrastat | 1.00% | 10 |
| 12 | Mixed Tocopherols (95%) | 0.50% | 5 |
| 13 | Transcutol | 2.50% | 25 |
| | Total | 100% | 1000 |

### Process of Preparation

The same process for preparation comprising preparation & mixing of phases was employed as described for microemulsion L, except for the hydrophilic surfactant Kolliphor RH 40 replaced by Polysorbate 80 in microemulsion II.

### Example 3: Microemulsion Composition II

TABLE 4
Composition of microemulsion III (1000 g batch)

| # | Component | w/w % | Mass (g) |
|---|---|---|---|
| 1 | Water | 59.17% | 591.7 |
| 2 | Citric acid | 0.35% | 3.5 |
| 3 | Na3-Citrate | 0.08% | 0.8 |
| 4 | PEG-40 | 13.6% | 136 |
| 5 | Captex 355 C | 10.0% | 100 |
| 6 | Capmul MCM | 10.0% | 100 |
| 7 | VitaSynol C | 2.0% | 20 |
| 8 | Vitamin E Acetate | 1.0% | 10 |
| 9 | D-Panthenyl Triacetate | 1.0% | 10 |
| 10 | SymDiol 68 | 1.0% | 10 |
| 11 | Spectrastat | 1.0% | 10 |
| 12 | Mixed Tocopherols (95%) | 0.5% | 5 |
| 13 | SLMW HA (Super Low- | 0.30% | 3 |
| | Molecular-Weight | | |
| | Hyaluronic Acid) | | |
| | Total | 100% | 1000 |

### Process of Preparation

Details of Phases in the Composition of microemulsion were similar as in Table 2, except for removal of the co-solvent Transcutol and the addition of Hyaluronic Acid. The same process for preparation comprising preparation & mixing of phases was employed as described for microemulsion I. Super Low-Molecular-Weight Hyaluronic Acid (SLMW HA) was added to the final mixture.

Out of the three microemulsion compositions I, II & III, disclosed above, the microemulsion I was subjected to various tests.

### Example 4: Particle Size Analysis and Stability Studies

4a—Particle Size Analysis

Particle size analysis was done using Zetasizer Nano ZS from Malvern Panalytical. The polydispersity index (PDI), which is a measure of the monodispersity of the sample, was 0.072. This indicates excellent uniformity and suggests that the microemulsion will have long-term stability.

4b—Stability Studies

The stability studies were performed as per the standard protocol. The accelerated in-house stability data showed that the microemulsion, as formulated, remained a stable microemulsion after continuous incubation at 40° C. for 3 months (which approximates a 12-month storage at 20-22° C. or room temperature (RT)). The microemulsion formulation also passed the 12-month room temperature stability test and remained stable for the entire test duration without any signs of instability.

TABLE 5
Stability study of microemulsion I

| Temperature | Duration | Condition |
|---|---|---|
| 20-22° C. (RT) | 12 months | Stable, Clear, (without discoloration or |
| | (365 days) | phase separation) |
| 40° C. | 3 months | Stable, Clear, (without discoloration or |
| | (90 days) | phase separation) |

### Example 5: Study on the Antibacterial Activity and Efficacy of Preservative in the Formulation

The efficacy of the preservative in the formulation and the microbial stability of the formulation was tested using the standard protocol as per PCPC microbiological guidelines method M-3 for water miscible personal care products, using the following test organisms:

### Test Organisms—

E. coli
P. aeruginosa
S. aureus
A. brasiliensis
C. albicans

### Procedure for PCPC M-3 Study:

1. The test microorganisms are prepared by growth in liquid or on agar culture medium. Microorganisms grown in liquid culture are centrifuged and washed prior to test.
2. Suspensions of test microorganisms are standardized by dilution in a buffered saline solution.
3. Test and control substances are dispensed, in similar known volumes, to sterile vessels
4. Independent volumes of Test and Control substances are inoculated with each test microorganism mixed and incubated. Control substances are immediately harvested and represent the concentration present at the start of the test, or time zero.
5. Incubated Test Substances are harvested at the conclusion of each contact time by chemical neutralization.
6. The number of surviving microorganisms at the respective contact times are assessed and logarithmic reductions are calculated based on initial concentrations observed at time zero.

Criteria of the study: A PCPC M-3 study to be considered scientifically defensible, the following criteria must be met:
1. The average number of viable test microorganisms recovered from the time zero samples must be approximately 1×10⁶ cells/ml or greater for bacteria and 1×10⁵ cells/ml or greater for fungi.
2. Ordinary consistency between replicates must be observed for the time zero samples.
3. Positive/Growth controls must demonstrate growth of appropriate test microorganism.
4. Negative/Purity controls must demonstrate no growth of test microorganism

### Passing Criteria

For bacteria, products must achieve at least a 99.9% reduction within 7 days following each challenge and show no increase for the duration of the test period. For yeasts and molds, products must achieve at least 90% reduction within 7 days following each challenge and show no increase over the duration of the test period.

### Study Results

The samples as per example 1 were tested for preservative efficacy per the PCPC Microbiological Guidelines method M-3 for water miscible personal care products and successfully met the passing criteria listed per PCPC Microbiological Guidelines.

### Example 6: Study to Evaluate Skin Irritation and Skin Sensitization of the Formulation

Formulation of example 1 was used for the study. The study was conducted on healthy volunteers who fulfilled the following inclusion criteria.

### Inclusion Criteria

1. Individuals who were not currently under a doctor's care.
2. Individuals who were free of any dermatological or systemic disorder that would interfere with the results, at the discretion of the Investigator.
3. Individuals who were free of any acute or chronic disease that would interfere with or increase the risk of study participation.
4. Individuals who completed a preliminary medical history form mandated by ALS and were in general good health.
5. Individuals who read, understood and signed an informed consent document relating to the specific type of study.
6. Individuals who were able to cooperate with the Investigator and research staff, and were willing to have test materials applied according to the protocol, and complete the full course of the study.

### Samples

Test materials to be tested under occlusive conditions were placed on an adhesive tape with paper filter discs with 1.0 cm2 (Adhesive tapes from 3M Company-Durapore (Code 1538) and Blenderm (Code 1525) or placed on an 8-millimeter aluminum Finn Chamber® (Epitest Ltd. Oy, Tuusula, Finland) supported on Scanpor® Tape (Norgesplaster A/S, Kristiansand, Norway) or an 8-millimeter filter paper coated aluminum Finn Chamber® AQUA supported on a thin flexible transparent polyurethane rectangular film coated on one side with a medical grade acrylic adhesive, consistent with adhesive used in state-of-the-art hypoallergenic surgical tapes or a 7 mm IQ-ULTRA® closed cell system which is made of additive-free polyethylene plastic foam with a filter paper incorporated (It is supplied in units of 10 chambers on a hypoallergenic non-woven adhesive tape; the width of the tape is 52 mm and the length is 118 mm) or other equivalents.

Test materials to be tested under semi-occlusive conditions were placed on an adhesive tape with paper filter discs with 1.0 cm2 (Adhesive tapes from 3M Company-Durapore (Code 1538) or placed on a test strip with a Rayon/Polypropylene pad or on a 7.5 mm filter paper disc affixed to a strip of hypoallergenic tape (Johnson & Johnson 1 inch First Aid Cloth Tape).

Test materials to be tested in an open patch were applied and rubbed directly onto the back of the subject.

Approximately 0.02-0.05 mL (in case of liquids) and/or 0.02-0.05 gm (in case of solids) of the test material was used for the study. Liquid test material was dispensed on a paper disk, which fit in the patch chamber.

### Procedure:

1. Subject individuals were requested to bathe or wash as usual before arrival at the facility.
2. Patches containing the test material were then affixed directly to the skin of the intrascapular regions of the back, to the right or left of the midline and subjects were dismissed with instructions not to wet or expose the test area to direct sunlight.
3. Approximately 0.02-0.05 mL of distilled water will be applied as a negative control. This was applied on an occlusive patch the first induction only.
4. Patches remained in place for 48 hours after the first application. Subjects were instructed not to remove the patches prior to their 48-hour scheduled visit. Thereafter, subjects were instructed to remove patches 24 hours after application for the remainder of the study.
5. This procedure was repeated until a series of eight (8) to nine (9) consecutive, 24-hour exposures had been made three (3) times a week for three (3) consecutive weeks.
6. Prior to each reapplication, the test sites evaluated by trained laboratory personnel.
7. Following a 10 to 14 day rest period a retest/challenge dose was applied once to a previously unexposed test site.
8. Test sites were evaluated by trained laboratory personnel 48 and 96 hours after application.
9. In the event of a reaction, the area of erythema and edema were measured. Edema is estimated by the evaluation of the skin with respect to the contour of the unaffected normal skin.
10. Subject individuals were instructed to report any delayed reactions that might occur after the final reading.
11. Any adverse reaction was immediately reported.

### OBSERVATION AND CONCLUSION

The test procedure was dermatologist tested and the under the study conditions, there was no indication of a potential to elicit dermal irritation or sensitization for the study formulation.

Thus, the micro emulsion prepared as per the present invention was found to be stable and was also resistant to microbial spoilage.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The embodiments were chosen and described in order to best explain the principles of the present invention and its practical application. It is understood that various omissions and substitutions of equivalents are contemplated as circumstances may suggest or render expedient, but such omissions and substitutions are intended to cover the application or implementation without departing from the scope of the claims of the present invention.

1. A cosmetic or pharmaceutical oil in water (o/w) microemulsion composition comprising,
a. a lipid phase comprising an oily component and one or more non-ionic surfactant(s) and one or more co-surfactant(s),
b. an aqueous phase comprising one or more non-ionic surfactant(s), and one or more co-surfactants; and
c. at least one co-solvent,
wherein the microemulsion has an average particle size of less than 100 nm.

2. The composition of claim 1 comprising,
a. a lipid phase comprising i) 5-20% w/w of an oily component, ii) 5-20% w/w of one or more lipophilic non-ionic surfactants;
b. an aqueous phase comprising i) 50-90% w/w of water, ii) 1-20% w/w of a one or more hydrophilic non-ionic surfactant;
c. 1-10% w/w of co-surfactants; and
d. 1-5% w/w of a co-solvent;
wherein the microemulsion has an average particle size of less than 50 nm.

3. The composition of claim 1, wherein the oily component is selected from fatty acid or polyol esters of animal or vegetable oil, C6-C12 medium chain triglycerides (MCT oil), caprylic/capric Triglyceride, or combination thereof.

4. The composition of claim 1, wherein the non-ionic surfactant of the lipid phase is selected from glyceryl caprylate/caprate, mono- and di-glyceride of medium chain fatty acids, polyglycerol fatty acid ester, caprylic/capric acid polyethylene glycol glyceride, or a combination thereof.

5. The composition of claim 1, wherein the non-ionic surfactant of the aqueous phase is selected from hydrogenated animal or vegetable oil and its derivatives, polyoxyl 40 hydrogenated castor oil, sorbitan fatty acid ester, polyoxyethylene sorbitan fatty acid ester, fatty alcohol polyoxyethylene ether, poloxamers, or a combination thereof.

6. The composition of claim 1, wherein the co-surfactants are selected from the group comprising of C8-C20 monoglyceryl ester, C8-C20 diglyceryl ester, C4-C8 1,2-alkanediols or a combination thereof.

7. The composition of claim 6, wherein the co-surfactant is C4-C8 1,2-alkanediol.

8. The composition of claim 7 wherein the co-surfactant C4-C8 1,2-alkanediol is selected from 1,2-butanediol (C4), 1,2-pentanediol (C5), 1,2-hexanediol (C6), 1,2-octanediol (C8), or a combination thereof.

9. The composition of claim 1, wherein the co-solvent is selected from low molecular weight mono- or poly-oxy-alkane diol comprising alkyl derivatives, selected from ethoxydiglycol, diethylene glycol monoethyl ether, diethylene glycol monomethyl ether, diethylene glycol mono-n-propyl ether, diethylene glycol mono-iso-propyl ether, diethylene glycol mono-n-butyl or mono-iso-butyl ether, diethylene glycol mono-n-hexyl ether, or a combination thereof.

10. The composition of claim 1, comprising at least one active ingredient in the range of 1% to 10% w/w based on the total weight of the composition.

11. The composition of claim 10, wherein the active ingredient is selected from Vitamin A and its derivatives, Vitamin B5 and its derivatives, Vitamin C and its derivatives, Vitamin E and its derivatives, Provitamins, Niacinamide, Hyaluronic acid, Ceramides, Benzoyl peroxide, Kojic Acid, Azelaic acid, Ferulic acid, Alpha-Hydroxy Acids (AHAs), Beta-Hydroxy Acids (BHAs), Polyhydroxy Acid (PHAs), Acetyl zingerone, Hydroquinone, other skin active agent, or a combination thereof.

12. The composition of claim 1, further comprising a preservative selected from medium chain mono- and di-glycerides of fatty acids, C6-C10 alkanediols, polyhydric alcohols, alkylhydroxamic acid or a salt thereof, or a combination thereof in a range of 0.5 w/w-5% w/w.

13. The composition of claim 1, may comprise one or more excipients selected from tonicity agent, anti-oxidant, stabilizing agent, buffering agent, or a combination thereof.

14. The composition of claim 1, wherein the pH of the composition is in the range of 4.0-7.0.

15. A method of preparing the cosmetic or pharmaceutical oil in water (o/w) microemulsion of claim 1 or claim 2, comprising the steps of:
a. preparing the lipid phase by mixing
i) the oily component selected from fatty acid or polyol esters of animal or vegetable oil, C6-C12 medium chain triglycerides (MCT oil), caprylic/capric triglyceride, or combination thereof;
ii) the non-ionic surfactant of the lipidic phase selected from glyceryl caprylate/caprate, mono- and di-glyceride of medium chain fatty acids, polyglycerol fatty acid ester, caprylic/capric acid polyethylene glycol glyceride, or a combination thereof;
iii) the oil soluble skin actives selected from Vitamin A and its derivatives, Vitamin B5 and its derivatives, Vitamin C and its derivatives, Vitamin E and its derivatives, Provitamins, Ceramides, Benzoyl peroxide, Azelaic acid, Ferulic acid, Beta-Hydroxy Acids (BHAs), Acetyl zingerone, other skin active agent, or a combination thereof;
b. preparing the aqueous phase by mixing
i) water;
ii) the non-ionic surfactant of the aqueous phase selected from hydrogenated animal or vegetable oil and its derivatives, polyoxyl 40 hydrogenated castor oil, sorbitan fatty acid ester, polyoxyethylene sorbitan fatty acid ester, fatty alcohol polyoxyethylene ether, poloxamers, or a combination thereof;
iii) the water-soluble skin actives selected from Vitamin B5 and its derivatives, Vitamin C and its derivatives, Niacinamide, Hyaluronic acid, Ceramides, Kojic Acid, Ferulic acid, Alpha-Hydroxy Acids (AHAs), Polyhydroxy Acid (PHAs), Hydroquinone, other skin active agent, or a combination thereof;
iv) the stabilizing and buffer agents;
c. combining the lipid phase, the aqueous phase,
d. adding to the mixture
i) the co-solvent selected from low molecular weight mono- or poly-oxy-alkane diol comprising alkyl derivatives, selected from ethoxydiglycol, diethylene glycol monoethyl ether, diethylene glycol monomethyl ether, diethylene glycol mono-n-propyl ether, diethylene glycol mono-iso-propyl ether, diethylene glycol mono-n-butyl or mono-iso-butyl ether, diethylene glycol mono-n-hexyl ether, or a combination thereof;
ii) the co-surfactant selected from C4-C8 1,2-alkanediols selected from 1,2-butanediol (C4), 1,2-pentanediol (C5), 1,2-hexanediol (C6), 1,2-octanediol (C8), or a combination thereof;
iii) the preservatives selected from medium chain mono- and diglycerides of fatty acids, C6-C10 alkanediols, polyhydric alcohols, alkylhydroxamic acid or a salt thereof, or a combination thereof
iv) the excipients selected from tonicity agent, anti-oxidant, stabilizing agent, or a combination thereof; and
e. stirring the mixture continuously to form an oil in water (o/w) microemulsion having pH 4.0-7.0 and an average particle size less than 100 nm.

16. The cosmetic microemulsion composition prepared by the method of claim 15.

17. A method of preparing the cosmetic or pharmaceutical oil in water (o/w) microemulsion of claim 2, comprising the steps of:
a. preparing the lipid phase by mixing
ii) the oily component selected from fatty acid or polyol esters of animal or vegetable oil, C6-C12 medium chain triglycerides (MCT oil), caprylic/capric triglyceride, or combination thereof;
iii) the non-ionic surfactant of the lipidic phase selected from glyceryl caprylate/caprate, mono- and di-glyceride of medium chain fatty acids, polyglycerol fatty acid ester, caprylic/capric acid polyethylene glycol glyceride, or a combination thereof;
iv) the oil soluble skin actives selected from Vitamin A and its derivatives, Vitamin B5 and its derivatives, Vitamin C and its derivatives, Vitamin E and its derivatives, Provitamins, Ceramides, Benzoyl peroxide, Azelaic acid, Ferulic acid, Beta-Hydroxy Acids (BHAs), Acetyl zingerone, other skin active agent, or a combination thereof;
a. preparing the aqueous phase by mixing
i) water;
ii) the non-ionic surfactant of the aqueous phase selected from hydrogenated animal or vegetable oil and its derivatives, polyoxyl 40 hydrogenated castor oil, sorbitan fatty acid ester, polyoxyethylene sorbitan fatty acid ester, fatty alcohol polyoxyethylene ether, poloxamers, or a combination thereof;
iii) the water-soluble skin actives selected from Vitamin B5 and its derivatives, Vitamin C and its derivatives, Niacinamide, Hyaluronic acid, Ceramides, Kojic Acid, Ferulic acid, Alpha-Hydroxy Acids (AHAs), Polyhydroxy Acid (PHAs), Hydroquinone, other skin active agent, or a combination thereof;
iv) the stabilizing and buffer agents;
b. combining the lipid phase, the aqueous phase,
c. adding to the mixture
i) the co-solvent selected from low molecular weight mono- or poly-oxy-alkane diol comprising alkyl derivatives, selected from ethoxydiglycol, diethylene glycol monoethyl ether, diethylene glycol monomethyl ether, diethylene glycol mono-n-propyl ether, diethylene glycol mono-iso-propyl ether, diethylene glycol mono-n-butyl or mono-iso-butyl ether, diethylene glycol mono-n-hexyl ether, or a combination thereof;
ii) the co-surfactant selected from C4-C8 1,2-alkanediols selected from 1,2-butanediol (C4), 1,2-pentanediol (C5), 1,2-hexanediol (C6), 1,2-octanediol (C8), or a combination thereof;
iii) the preservatives selected from medium chain mono- and diglycerides of fatty acids, C6-C10 alkanediols, polyhydric alcohols, alkylhydroxamic acid or a salt thereof, or a combination thereof
iv) the excipients selected from tonicity agent, anti-oxidant, stabilizing agent, or a combination thereof; and
d. stirring the mixture continuously to form an oil in water (o/w) microemulsion having pH 4.0-7.0 and an average particle size less than 100 nm.

18. The cosmetic microemulsion composition prepared by the method of claim 16.
